(19) United States
(12) Patent Application Publication
(10) Pub. No.: US 2021/0374626 A1
(43) Pub. Date: Dec. 2, 2021

(54) Network-Based Medical Patient Servicing System

(71) Applicant: David Reyes, Hollister, CA (US)

(72) Inventor: David Reyes, Hollister, CA (US)

(21) Appl. No.: 17/403,621

(22) Filed: Aug. 16, 2021

Related U.S. Application Data
(63) Continuation of application No. 16/375,682, filed on Apr. 4, 2019.
Application No. 16/375,682 is a continuation of application No. 13/466,574, filed on May 8, 2012, now abandoned.
(60) Provisional application No. 61/614,332, filed on Mar. 22, 2012.

(51) Int. Cl. G06Q 10/06; A61B 5/00; G16H 40/20; G16H 40/67

(52) U.S. Cl. CPC G06Q 10/06; A61B 5/0022; A61B 5/01; G16H 40/67; G16H 40/20

(57) ABSTRACT

A system has a computerized connected to the Internet network, a data repository, a display monitor, a portable communication device having audio and imaging apparatus, connected to the Internet network, located at a at a patient's geographic location, an interactive interface displayed on the display monitor, and icons displayed in a queue in the interactive interface, each icon representing an appointment, each icon comprising indicia providing identification of the patient, date and time of day for the associated appointment, and real-time status of the appointment, as ready and waiting, in progress or finished. A physician user at the computerized appliance, by virtue of execution of the software by the processor, is enabled to interact with an individual one of the icons indicated as a waiting appointment, such that selecting the icon initiates a video conference between the computerized appliance and the portable communication device.

### CROSS-REFERENCE TO RELATED DOCUMENTS

The present application is a continuation of co-pending application Ser. No. 16/375,682, filed Apr. 4, 2019, which is a continuation of application Ser. No. 13/46,574, filed May 8, 2012, which claims priority to provisional patent application, 61/614,332, filed Mar. 22, 2012, entitled “Remote Delivery of Physician-Directed Medical Services”. Disclosure of the parent applications is incorporated herein in its entirety at least by reference.

### BACKGROUND OF THE INVENTION

### 1. Field of the Invention

The present invention is in the field of medical services including delivery of services over a network and pertains particularly to methods and apparatus for serving remote-located patients through a network interface.

### 2. Discussion of the State of the Art

In the field of medical services, patients currently in need of medical assistance come to a doctor's office, to an emergency room, or to an urgent care center where a physician on duty may examine, diagnose, and treat the patient's medical problems or conditions. This service model, necessitated traditionally in part by a need for expensive and sometimes cumbersome equipment, is a “horizon-limited” model, wherein a physician may serve a clientele in a limited geographic region. For example, a primary-care physician in a specific region will typically have clientele domiciled within that general region. Other types of physicians that specialize in chronic illnesses such as cancer may see patients that are not from the local region and therefore must travel sometimes significant distances to make a scheduled appointment or several such appointments and thus incur relative expenses such as gas, hotel, and like expenses.

More recently, and with the advance of wide-area-network connectivity such as the Internet, physicians who are typically specialists in some types of surgery, or specializing in oncology services with access to appropriate telecommunications and multimedia equipment have conducted remote teleconference sessions including multimedia with certain patients who may be in need of such services.

The inventor is aware of a U.S. Pat. No. 7,912,733 issued on Mar. 22, 2011 to Leon M. Clements as first-named inventor, hereinafter referred to as Clements. Clements proposes a remote medical services delivery suite that enables patients having scheduled medical appointments to travel to local or near-by regional medical clinics that are equipped with remote patient services work stations where the patients, with the aid of an onsite registered nurse (RN) or other medically trained staff, may connect to and receive remote physician services from an off-site physician operating from a specialized physician workstation.

One challenge with the system provided by Clements is a requirement that patients must travel to or be a resident of a local or near-regional medical facility equipped with the required multimedia apparatus and network-access facilities in order to participate in the system. Moreover, physician's facilities are required that have specialized physician workstations including full multimedia capabilities. One or more specialized medical access servers are provided and deployed on the network in the regions that are serviced in order to enable successful servicing of patient's medical needs in Clements.

Another limitation of the system of Clements is a requirement that certain non-portable medical evaluation equipment is required to be maintained onsite in specialized patient clinics where the patients must travel to access services. Other limitations exist in the system of Clements as well, which will become apparent to the skilled artisan considered in the light of the present invention.

Therefore, what is clearly needed is a network-based medical patient servicing system that solves the problems mentioned above. Such a system would increase access to immobile or travel-limited patients in a manner not dependant on location and stationary equipment.

### SUMMARY OF THE INVENTION

In one embodiment of the invention a system is provided, comprising a computerized appliance having a processor executing software, the computerized appliance connected to the Internet network, a data repository coupled to the computerized appliance, a display monitor coupled to the computerized appliance, a portable communication device having audio and imaging apparatus, connected to the Internet network, and located at a at a patient's geographic location remote from the computerized appliance, an interactive interface displayed on the display monitor by execution of the software by the processor, and a plurality of icons displayed in a chronological queue in the interactive interface, each icon representing an appointment with a patient, each icon comprising first indicia providing identification of the patient, second indicia providing date and time of day for the associated appointment, and third indicia providing real-time status of the appointment, as ready and waiting, in progress or finished. A physician user at the computerized appliance, by virtue of execution of the software by the processor, is enabled to interact with an individual one of the icons indicated as a waiting appointment, such that selecting the icon initiates a video conference between the computerized appliance and the portable communication device and updates the status of the appointment to in progress.

In one embodiment the physician user, engaged in a video conference with the portable communication device at the patient's geographic location, is enabled to access medical records associated with the patient, and to display such records on the display monitor. Also, in one embodiment the patient's medical records are downloaded from an Internet site. Also, in one embodiment the physician user is enabled, through the software execution, to rearrange the appointment icons as to date and time of day. And in one embodiment the indicia providing status is a tag having a color indicating the status.

In one embodiment the system further comprises a local area network (LAN), the computerized appliance being a first appliance connected to the LAN, further comprising second computerized appliances with display monitors also connected to the LAN, enabling a plurality of physician users to conduct video conferences with second portable communication devices at patient's geographic locations. Also, in one embodiment a medical assistant is sent to the patient's geographic location near the time of an appointment, and the medical assistant operates the portable communication device in a video conference with a physician user. In one embodiment the system further comprises a medical measuring device coupled to the portable communication device, the medical measuring device measuring a physical characteristic of the patient and communicating the measurement to the computerized appliance over the Internet network. In one embodiment the computerized appliance is one of a smart phone, a laptop computer, a notebook computer, or a pad device. And in one embodiment the system further comprises functionality enabled by execution of the software enabling persons operating Internet connected computer devices to access the computerized appliance over the Internet network, and to request or cancel an appointment.

In another aspect of the invention a method is provided, comprising displaying a plurality of icons in a chronological queue in an interactive interface on a display monitor coupled to a computerized appliance connected to the Internet network and having a coupled data repository, each icon representing an appointment with a patient, with each icon comprising first indicia providing identification of the patient, second indicia providing date and time of day for the associated appointment, and third indicia providing real-time status of the appointment, as ready and waiting, in progress or finished, and interacting with an individual one of the icons by a physician user, initiating a video conference between the computerized appliance and a portable communication device having audio and imaging apparatus, located at a geographic location of the patient remote from the computerized appliance, initiating a video conference between the computerized appliance and the portable communication device and updating the status of the appointment to in progress.

In one embodiment of the method the physician user, engaged in a video conference with the portable communication device at the patient's geographic location, accesses medical records associated with the patient, and displays such records on the display monitor. Also, in one embodiment the method comprises downloading the patient's medical records from an Internet site. In one embodiment the method comprises the physician user rearranging the appointment icons as to date and time of day. And in one embodiment the method comprises providing status of an appointment by a tag having a color indicating the status.

In one embodiment the method further comprises enabling a plurality of physician users to interact with appointment icons and initiate video conferences by connecting a plurality of computerized appliances having connected displays and data repositories to a local area network (LAN). In one embodiment the method comprises sending a medical assistant to the patient's geographic location near the time of an appointment, the medical assistant operating the portable communication device in a video conference with a physician user. In one embodiment the method further comprises coupling a medical measuring device to the portable communication device, measuring a physical characteristic of the patient by the medical measuring device and communicating the measurement to the computerized appliance over the Internet network. In one embodiment the computerized appliance is one of a smart phone, a laptop computer, a notebook computer, or a pad device. And in one embodiment the method further comprises accessing the computerized appliance over the Internet network by a person operating an Internet connected computer device, requesting or cancelling an appointment.

### BRIEF DESCRIPTION OF THE DRAWING FIGURES

FIG. 1 is an architectural overview of a patient-servicing network according to an embodiment of the present invention.

FIG. 2 is an example of an interactive interface to be used by a physician in an embodiment of the present invention.

FIG. 3 is a process flow chart depicting steps for obtaining appointments and servicing those appointments according to an embodiment of the present invention.

FIG. 4 is an exemplary screen shot of the electronic interface of FIG. 1 including appointment and third-party session queues according to an embodiment of the present invention.

FIG. 5 is a process interaction chart depicting patient services interaction according to a further embodiment of the present invention.

### DETAILED DESCRIPTION

The present inventors provide a medical patient servicing system that enables patients who are logistically remote or challenged, to participate in physician-mediated appointments without leaving their residential premise. The present invention is described in enabling detail using the following examples, which may describe more than one relevant embodiment falling within the scope of the present invention.

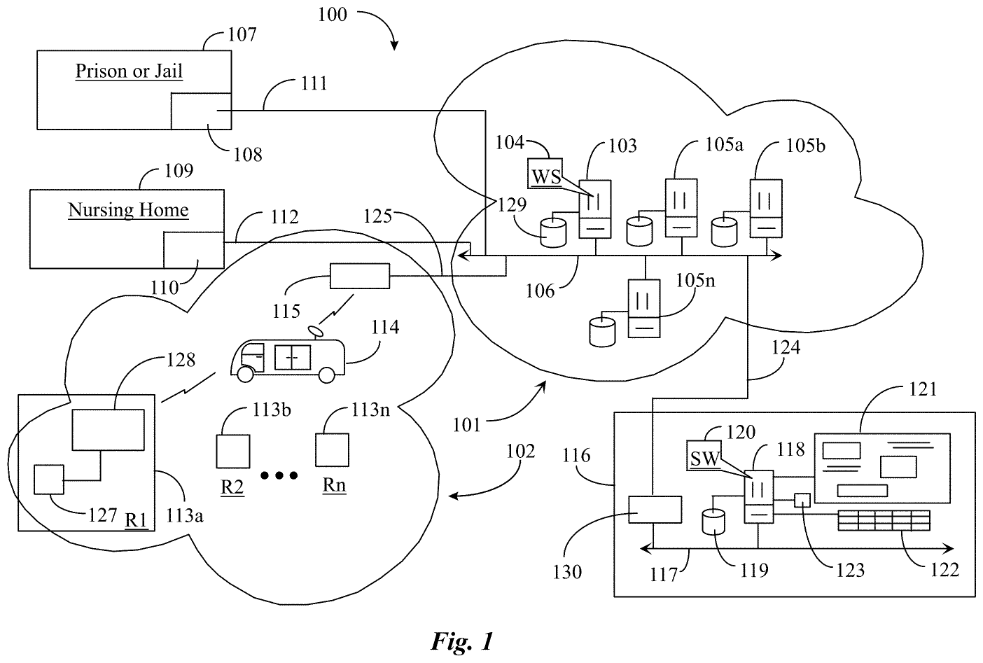

FIG. 1 is an architectural overview of a patient-servicing network 100 according to an embodiment of the present invention. Service network 100 includes an Internet network 101 and a connected sub network 102, which may be a wireless network such as a wireless fidelity (WiFi) network. Internet network 101 is further characterized by an Internet backbone 106. Internet backbone 106 represents all of the lines, equipment, and access points that make up the Internet network as a whole including connected sub-networks. Therefore, there are no geographic limitations to the practice of the present invention.

Internet backbone 106 supports a Web server 103. Web server 103 includes a processor and a connected data repository 129. Server 103 includes a processor and software executing on the processor from a non-transitory medium, the software having access to all of the data required to enable function as a Web server. Server 103 is adapted to serve Web pages and may be maintained by a third-party Web-hosting service. In this example, a Website (WS) 104 is hosted on server 103. Website 104 is an access point for initiating services of the present invention.

Other Web servers or mirror servers are illustrated in this example including Web servers 105a, 105b, and 105n. Each Web server 105 (a-n) includes a processor (not explicitly illustrated) and a connected data repository. Web servers 105 (a-n) are assumed in this example to include software executing from their respective processors from a non-transitory physical medium, the software having access to all of the data and instruction required to function as a Web server. WS 104 may be served upon request on server 103 and or any one of or any combination of servers 105 (a-n). Backbone 106 carries Internet inter-communication between server 103 and other Internet-connected servers such as servers 105 (a-n). The servers illustrated herein represent the plethora of servers in the Internet that host private, public, social, commercial, and government Websites that provide data, functions and a broad variety of services.

An institution 107 illustrated in this example is in one instance a prison or jail facility. Facility 107 may be any type of housing provided for holding institutionalized persons or inmates. Facility 107 represents one of a large number of prisons, jails, or like institutions both in the US and around the world. Facility 107 is typically managed under a government jurisdiction or department. The government jurisdictions overseeing facilities like facility 107 are required to provide medical services for the inmates in many or most cases. Such facilities may include one or more common areas 108 where inmates undergoing in-house medical treatments may be transported for the purpose of obtaining medications, exams, or other procedures that might be performed on site such as by a nurse or physician. Area 108 may be a waiting area with one or more exam rooms where patients may be processed for medical exams, prescriptions, dental services and the like. Area 108 is not required to have resident medical equipment or physician-trained personnel in order to practice the present invention. Where such facilities like facility 107 have medical equipment, and personnel, such apparatus and personnel may be leveraged or not leveraged in conjunction with practice of the present invention.

An institution 109 is illustrated in this example as a nursing home. Facility 109 may be an assisted living community, a mental institution, or any other facility that houses disabled, retired, or elderly persons and provides these persons with living arrangements. Persons residing at facility 109 may be limited in their mobility and transportation options. Facility 109 may include one or more common areas 110 where residents undergoing in-house medical treatments may be transported for the purpose of obtaining medications, exams, or other procedures that might be performed on site such as by a nurse or physician. Area 110 may be a waiting area with one or more exam rooms where patients may be processed for medical exams, prescriptions, dental services and the like. Area 110 is not required to have resident medical equipment or physician-trained personnel in order to practice the present invention. Where such facilities like facility 109 have medical equipment, and personnel, such apparatus and personnel may be leveraged or not leveraged in conjunction with practice of the present invention.

A number of private residences, R1-Rn, also characterized by element numbers 113 (a-n) with private residence 113a expanded for view. Private residences 113 (a-n) represent literally millions of such personal domiciles in the US and worldwide, where families reside. Private residences may also be duplexes, apartment buildings, or condominium buildings containing a number of private residences. In a preferred aspect of the present invention, one or more non-physician medical assistants may be dispatched to various locations as determined by location information in a schedule for appointments, which are preset by patients requiring medical evaluation or assistance, or by persons acting on behalf of those patients. As such, appointments that facilitate remote physician mediation are processed onsite according to location information of each appointment. Non-physician assistants may be medical assistants (MAs) having at least some medical training. A registered nurse (RN) or an emergency medical technician (EMT) might be qualified to function as a non-physician assistant in this example.

For facilities like facility 107 and facility 109, appointments may be held in common areas 108 and 110 respectively. Such appointments may also be held in individual cells, rooms, apartments, suites, or other living arrangements within each facility. For private residences, appointments are held within the residences on location or within a medically equipped mobile unit, which in this example is a van 114. One or more non-physician assistants may drive van 114 to appointment locations such as residence 113a. In one embodiment, van 114 may also respond to facilities like facility 107 and facility 108. Van 114 is medically equipped in a preferred embodiment and also equipped with all of the networking equipment and software to enable wireless access to Internet 101.

In this example, facility 107 and facility 109 both have connectivity to Internet 101, more specifically to backbone 106 through Internet access lines 111 and 112 respectively. Where such facilities include adequate medical equipment and Internet access capability, non-physician assistants may not require a medically equipped van like van 114. Residences typically do not contain adequate medical equipment and may not have adequate or any Internet connectivity. Therefore, van 114 may be used to provide both medical equipment and supplies, but also Internet and communications capabilities for interfacing with a remote physician during location-based appointments. Van 114 represents all types of medically equipped vehicles that may provide communications interfaces and Internet access for at least the non-physician assistants arriving at individual domiciles such as residences 113 (a-n) or at facilities 107 and 109.

In this example, van 114 brokers a wireless Internet access by providing mobile wireless fidelity (WiFi) so that all appointments are conducted over an Internet connection. Van 114 in many embodiments is equipped to provide a local Wi-Fi system to engage with portable appliances in the vicinity of the van, and to provide broadband Internet access through one or more base stations 115 supported by Internet backbone 106. Base station 115 may connect to the Internet backbone through network access line 125 in any of a wide variety of known techniques.

In FIG. 1 a medical device 127 is illustrated connected to a network-capable appliance 128. Peripheral medical device 127 may be one of an imaging device, an acoustic measurement device, a blood pressure measurement device, a body temperature measurement device, a weight measurement device, or other device. In addition to a processor and connected or internal data repository, network-capable appliance 128 includes an adequate display screen or monitor, a means for data input, and a networking card or circuitry enabling Internet connection through van 114 or some other access point such as a pre-existing broadband network. Network-capable appliance 128 is brought to appointments and is operated by one or more non-physician assistants.

It is important to note herein that one or more network-capable appliances may be brought to onsite appointments and may be leveraged for conducting remote wireless appointment sessions between patients and remote physicians. Network-capable appliance 128 may be one of a laptop computer, a notebook computer, an iPad, or a portable desktop computer. In a preferred embodiment, appliance 128 is adapted to dock or otherwise connect to one or a number of different peripheral medical devices 127. Many medical procedures may be performed with relatively simple devices such as blood pressure cups, diabetes testing devices, ultrasound devices, blood oxygen measurement devices, skin analysis devices, and the like.

Such current and future envisioned peripheral devices may leverage powerful computing platforms and displays of appliances like network-capable appliance 128. Connection between a peripheral medical device and a network-capable appliance may be achieved through such as universal serial bus (USB) docking (wired or wireless), serial port connection, or other adapters for providing digital paths between the device and appliance including power from the host device. Likewise, many peripheral devices might be powered independently and may leverage the network-capable appliance solely for collecting and uploading medical data taken by such a device during an examination for example. In one example, advances in imaging software have enabled an application for imaging the skin of a patient through the camera function of a device like an Android device or iPhone. Therefore, in many instances applications for working with peripheral devices and for on-board devices may be provided to reside and execute from a non-transitory medium on the host-device processor.

In this example, non-physician assistants may bring appliances and peripherals into residences as illustrated at residence 113a, or patients may enter van 114 to conduct a medical appointment. Moreover, non-physician assistants may bring network-capable appliances and peripheral medical devices into the common areas of facilities 107 and 109. In this variation, several appointments may be run concurrently at the facilities where a number of patients are scheduled for appointments on a same day and each patient comes to the common area for their appointments. Patients may still enter van 114 for an appointment if the van is available at the location such as may be the case with med-van outreach services for the homeless, for example. Otherwise, wireless connectivity of each facility and in-house medical equipment might be utilized in addition to non-physician assistant ported appliances and medical peripheral devices without departing from the spirit and scope of the present invention. In more robust embodiments the non-physician assistant may have considerable portable equipment for medical assessment, and such equipment may communicate via a provided WiFi from van 114, or the non-physician assistant may use an appliance that connects to the WiFi to which such equipment may connect and communicate. There are many possibilities.

A central facility 116 is illustrated in this example and may be a single room or a multi-tenant facility having a Local Area Network (LAN) 117 connected to Internet 101 through a modem 130 through Internet access line 124. There are a variety of ways familiar to a skilled person that this connection and communication may be implemented. LAN 117 supports at least one computerized appliance 118, typically a LAN-connected workstation or other type of network-capable appliance that is operated by one or more physicians. Appliance 118 includes a processor (not illustrated) and a data repository 119. Data repository 119 contains patent information, medical chart information, and other data deemed important to reference or access during a remote appointment session.

LAN-connected appliance 118 further includes a display monitor or screen 121, an input mechanism 122 such as a keyboard, and an output mechanism such as a back-up memory or storage device 123. Appliance 118 may execute software 120 from a non-transitory physical medium connected to or residing on the processor of the appliance.

SW 120 is adapted to drive an electronic interface 126. Electronic interface 126 is executed and running during the physician's shift to enable the physician to mediate onsite appointments facilitated by the one or more non-physician assistants. Appliance 118 executing software 120 provides and drives the electronic interactive interface 126, illustrated in display monitor 121.

Non-physician assistants such as an MA, for example, may arrive at a facility or residence in an equipped van such as van 114 for example. In a minimalist embodiment there may be no equipped van, but a medical assistant may arrive with a hand-held device, such as an iPad™, and one or more instruments such as equipment to measure blood pressure, temperature, heart rate, and the like, to provide data for vital signs for the physician's use. This equipment may connect directly to the iPad™ as described further above, or there may be a program executing on the iPad™ for the MA to manually enter data to be communicated to server 103 and thence to the doctor operating at central facility 116. An important piece of equipment for the MA is image-capture and ability to communicate images, either as still images or preferably as video, to Internet server 103 and subsequently to a physician using interface 126 at central facility 116.

In one embodiment of the present invention, SW 120 executes from an Internet-connected server and both physicians and non-physician assistants have access to the interface by connecting to the Internet and Website 104. In another embodiment, software 120 runs on the physician's workstation or appliance 118 and is accessed by the medial assistants at each scheduled appointment time. In a preferred embodiment, physicians and assistants enjoy full bi-directional communication through text, video, and sound. Files may be transferred back and forth over the network in real time. Devices and assistant input at the patient's end provide the physician with adequate exam data. Charts and other records may be served or imported into electronic interface 126.

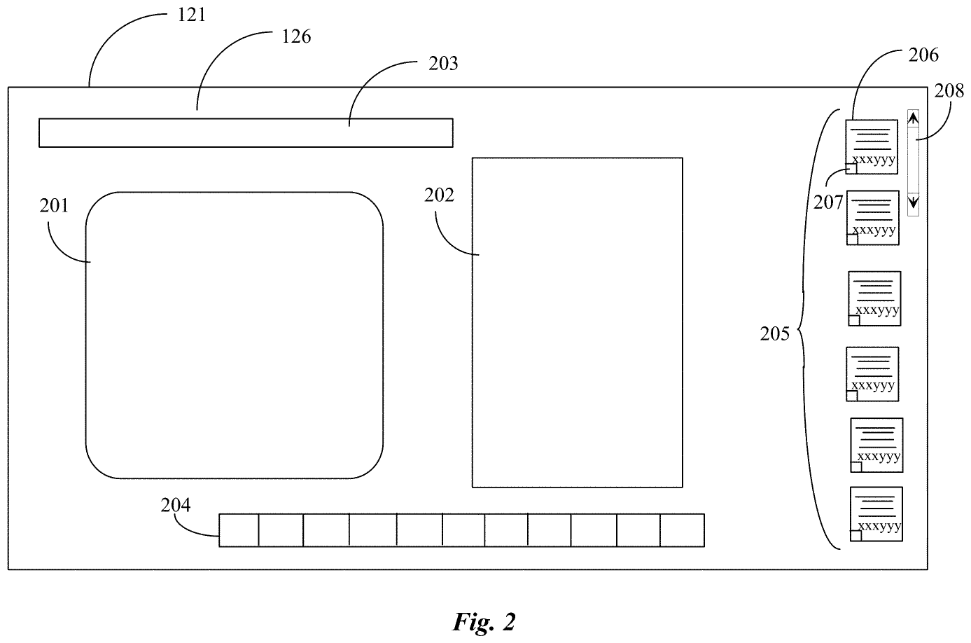

FIG. 2 is an example of an interactive electronic interface 126 usable by a physician in an embodiment of the present invention. Electronic interface 126, in display on screen 121, is personalized to the attending physician in a preferred embodiment and functions as the attending physician's command center. The physician may interact with, diagnose, and treat patients in a selectable order. Electronic interface 126 is an interactive interface implemented with a plurality of interactive links and or buttons that enable initiation of various interactive functions. The physician may interact with electronic interface 126 via touch screen, keyboard and a pointer device, voice input, or other supported input technologies.

Interface 126 includes a header bar 203 that may display an enterprise logo, enterprise information, names of physicians, and other medical professionals associated with the enterprise, emergency medical services and the like. A video window 201 is provided for real-time video conferencing with patients and medical assistants. Electronic interface 126 includes a data display window 202. Window 202 may display data, both graphic data such as patient imaging records and text, such as patient chart data, email, messaging, alerts, and the like. For example, window 202 might display a patient's medical chart while a medical session is in progress, as needed. A menu bar 204 provides a plurality of interactive links, from which a physician may select to link to and use textbooks, medical journals, case studies, medical dictionaries, a calculator, and to activate a browser to browse Internet content for further information as may be needed in a medical session.

It will be apparent to the skilled artisan that electronic interface 126 may support a single interactive window for displaying data, video with sound, or two or more interactive windows without departing from the spirit and scope of the present invention. Universal data display windows (window assigned to contain display multiple different types of data) such as windows 201 and 202 may also be detached from their anchors and moved to another location, or to “float” outside of the visible boundary of the interface. Sessions may be conducted through such windows and multiple views, of different aspects of a session may be presented and displayed simultaneously and moved to any location on the physical screen 121. Windows 201 and 202 may also be minimized or expanded in display. Session data such as real time imaging data, video with sound, sequential slides in a slide show, live teleconference, may be recorded automatically as it is received and paused or play back for review at the direction of the attending physician.

In practice of the invention persons in need of medical attention, or other persons such as caregivers and institutional personnel may schedule an appointment for a medical session with a physician associated with the enterprise. These appointments such as appointment 206, may be made through a publicized website such as website 104 (FIG. 1). Active appointments, as well as, pending and completed appointments represented collectively herein as indicia 205 may be physically characterized by interactive boxes, windows, or icons displayed within electronic interface 126. In one embodiment an icon 205 may list some information as text, such as the patient's name, gender and age, and the date and time for the session. In one embodiment, each appointment representation such as appointment 206 may include a visible indicator such as a flag 207.

The flags may be in color, such that Green, which may indicate, in certain embodiments, that the non-physician assistant such as an MA is ready to initiate a session with the physician. The physician may click or double-click on the icon to establish or accept the session. A yellow flag may be initiated to replace the green flag indicating that an established session is on hold. The physician may return to a session placed on hold by a single click or keyboard stroke action. A red flag might be used in certain embodiments to indicate a higher priority session. Flags may change color in an automated fashion or be initiated to change color manually.

Color changes to flags associated with active appointments might be communicated in one of several ways by a non-physician assistant or MA, such as by requesting a color change through a mobile client application executed to run from a non-transitory medium on a processor coupled or otherwise integrated with a portable or handheld network-capable appliance carried to the location of the appointment such as appliance 128 of FIG. 1 (application not illustrated). In one embodiment, where a client application is provided, the non-physician assistant may manually implement a color change (where allowed). A flag exhibiting or set to transparency or no color may be used to indicate that an appointment session is completed, has been or requires rescheduling, or is currently scheduled for that clinic day but not yet pending on the appointment calendar. Another color may be used to indicate that a patient and MA are in a virtual waiting room or queue, but not yet engaged. In another embodiment, a color such as orange may indicate that a patient is currently under exam by the non-physician assistant for vitals, the results of which may be immediately forwarded to the attending physician just before the physician accepts the active session. Tasks that do not require physician oversight are completed before the physician accepts the appointment.

Appointment indicia 205 may appear automatically in interface 126 such as by automated data import function. Indicia 205 may present within a virtual queue analogous to a telephony queue where additional data to the patient is available such as estimated waiting time for physician participation, and so on. Initiation or selection of an appointment such as appointment 206 will cause any live video feed sourced from the location of the patient and MA to be displayed in window 201. The MA, patient and the physician may interact through live two-way video call, or video chat. Prior to the beginning of an active live session, the physician may retrieve, or otherwise be served a patient's current medical chart detailing all of the latest medical information about the patient and display it in a data presentation window such as 202 to familiarize with the state of the patient before the live session.

During an active session the doctor may switch to current vital signs in window 202, and there may be an input for the doctor to switch between the two. Alternatively, both may be exhibited in a portion of window 202 and the doctor may resize them and scroll each as well. Moreover, additional windows containing such data may be launched through interaction with the electronic interface. A physician mediating a live session may also access further information, such as from case studies or web sites by using interactive links in menu bar 204 as may be required. The physician may, as a session progresses, query the patient, ask the MA to perform certain procedures to provide further information, such as asking the MA to zoom in or re-focus the image device at the patient's end to examine a wound, for example.

One or more links available within menu bar 204 may cause requested electronic forms like receipts, prescriptions, medical work orders, or the like to pop-up in display or in their own windows for physician use in prescribing medications, performing evaluations, making recommendations, etc. Such forms, where required, may be digitally signed by the physicians and electronically received by the non-physician assistant. The physician may also interact with third parties through interface 126 such as with other physicians, specialists, pharmacists, radiologists, and other parties as may be required.

In one embodiment, there may be one or more links on tool bar 204 or otherwise within interface 126 for dispatching an emergency vehicle to take a patient to a hospital emergency room. At the end of a session the doctor may update the patient's medical chart and provide specific instructions and “doctor's orders” for the patient, which may be printed out by the MA using local equipment carried either by the MA or in van 114. In a preferred embodiment, all procedure and activity of a live session is recorded and saved with all demographic info for easy cross-reference and retrieval in future appointments or as may be required by professionals on a team working with the patient. Such recorded data may be stored in repository 129 at server 103 in a database, which may be made available to the physicians on duty and other persons and institutions authorized to see and use the information. In one embodiment, patient records and recorded session data including patient record updates may be stored on a cloud computing network such that the information may be backed up in two or more repositories. Such services may be provided by a third-party without departing from the spirit and scope of the present invention.

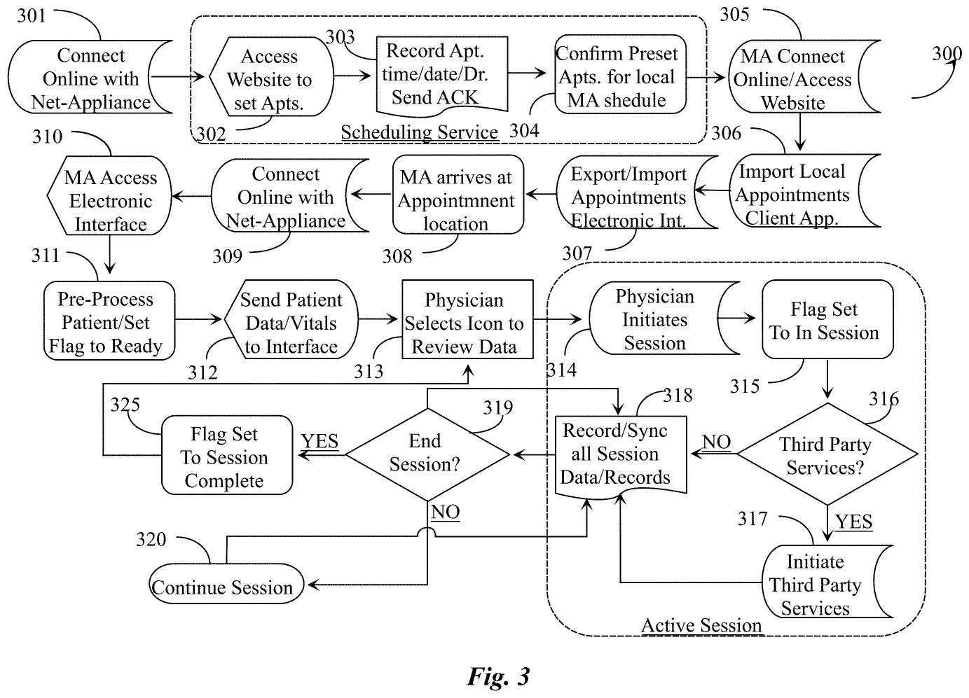

FIG. 3 is a process flow chart 300 depicting steps for obtaining appointments and servicing those appointments according to an embodiment of the present invention. At step 301, a patient or a person acting on behalf of the patient such as a family member, or caretaker may connect online such as on the Internet network using a network-capable appliance. The network-capable appliance may be one of a cellular telephone, a laptop computer, a notebook computer, an android device, an iPad, or a portable computer. Once connected, the patient or other person authorized to schedule an appointment on behalf of the patient, navigates to and accesses a Website (WS) at step 302. The Website of step 302 is analogous to Website 104 on server 103 of FIG. 1 to schedule one or more appointments. Registration services may be performed for the patient if the patient is not already registered and authorized to use the Website service for scheduling appointments.

At step 303, Web services record the requested appointment time and date, along with other appointment particulars such as the attending physician or physicians, the reason for scheduling the appointment, for example, initial exam, follow up evaluation, doctor consultation, etc. The scheduling function of the Web service performs a lookup in a scheduling database analogous to a database in repository 129 of FIG. 1 to access available appointment times according to the particular requirements of the session. A patient or person acting on behalf of that patient may access the Website from any network access point including from home, from work, while traveling, or from any location providing Internet access. Patient identification and authentication information may be required to set an appointment.

In one embodiment, patients may set appointments without registration or authentication procedures. These services may be performed for the patient at a first session and can be used to authenticate the patient for further appointments. After setting an appointment, the system automatically sends an acknowledgement (ACK) back to the network appliance used to set the appointment. Moreover, any pre-appointment information particular to the medical service sought may be sent back with the acknowledgement. For example, a first session may require that a patient or caregiver prepare a list of current medications and supplements. Other such pre-appointment requirements may be communicated at the time of setting of the appointment.

At step 304, some time after the appointment is set but before the date and time of the appointment, the Web service may contact the patient and, or caretaker that set the appointment to confirm the date and time of the appointment to ensure the patient will be available for remote session medical services. The patient or caregiver may have the option of canceling the appointment and may set a new appointment. Confirmation may be made by alert, message, telephone call, or other communication methods. In one embodiment, the Web service may additionally add the appointment to a calendar-based alert system to give the patient or caregiver a reminder before the scheduled time and day of the appointment. In one embodiment, scheduling and appointment confirmation is performed by a third-party contact service. In another embodiment the service is an integrated part of the physician's services and may be performed by onsite receptionists, or other doctor staff. Third party provisioning expands the opportunity to reach more patients and to schedule work for more physicians from private practice offices and from facilities belonging to different provider networks.

At step 305, a non-physician assistant such as a medical assistant (MA) may connect online using any network-capable appliance and access the Website of step 302 before the day appointments made through the site are scheduled. The MA is charged with traveling locally to patient locations who have appointments scheduled on that day. As described previously, there may be one or more than one MA that travels to an appointment location, for example, to facilitate remote appointment sessions. At step 306, the MA may import locally set appointments into a client application running on a hand-held network-capable appliance used by the MA. The MA may also import preset and confirmed appointments into other applications running on any network-capable appliance. In a preferred embodiment the appliance is portable or mobile such as an iPad, smart phone, or other such device.

The MA imports all of the appointments that can reasonable are met relative to the MA traveling to and arriving at the patient location or facility where patients are scheduled. Rules may be provided to enable system estimation of travel time, expected session length, and the like to ensure the MA does not have too many appointments to meet all of them. The appointments are confirmed with the patient or caregiver to reduce the risk that the MA will travel to a location and find that the appointment is cancelled or that the patient is not there.

The client application running from a non-transitory medium associated with the processor of the network-capable appliance used by the MA may include further provisions for accessing GPS assisted directional services so the MA may map all of the day's appointment locations and get directions to and from each appointment. In one embodiment, messaging may be used to communicate to patients and caregivers if an MA, for example, is running behind schedule for any one appointment location. Once the MA has all of his or her appointments to run for the day, the same appointment list may be simultaneously exported or imported into a physician-centric electronic interface such as interface 126 of FIG. 2 at step 307. In this way, a physician has notification of the appointment calendar for that day. In this case, the appointments may be entered into an appointment queue visible in the electronic interface used by the physician to establish the remote sessions.

It is noted herein that if the number of appointment locations for a day are located at a facility such as a jail or nursing home, then the system may account for less travel time for the MA and may allow more appointments to be scheduled for that day. In one embodiment, travel optimization is performed at the Website for a number of preset appointments for an MA at the time of scheduling or during confirmation. Such optimization may suggest a different order of appointments relative to the chronology of appointment times to reduce redundant travel routes for the MA and to potentially add one or more appointments to the workload. A third-party satellite mapping service that the MA has access to may perform travel optimization before or at the onset of travel to the scheduled appointment locations.

In step 308, the MA arrives at an appointment location. The location may be at a facility such as a jail, or nursing home, or at a private residence such as a home or apartment. In one embodiment, persons traveling may schedule and receive services at a hotel room, airport, or other public locations. The MA may arrive to an appointment location in a medically equipped van that also provides connectivity services to the Internet such as wireless fidelity (WiFi) services.

It is noted herein that more than one MA may be traveling together running the same appointment calendar without departing from the spirit and scope of the present invention. The MA may arrive to an appointment location that already has wireless Internet connectivity without a special van or vehicle. One or more MAs might carry hand-held or portable network-capable appliances and portable medical equipment adapted to work with those appliances to appointment locations without requiring a van or other special vehicle. Appointments may be carried on wherever there is a good access to the network. In one embodiment, Internet connectivity state is assessed of the patient location at the time the appointment is set giving the system ample time to determine if a network-access Van will be required at one or more locations.

At step 309, the MA connects online with a network-capable appliance and or other portable equipment such as video and sound equipment that may or may not be integrated with the particular appliance used to access the Internet. At step 310, the MA accesses the physician-centric electronic interface that includes an appointment queue or calendar containing the preset and confirmed appointments set for the MA and remote physician. The MA may, in one embodiment, preprocess the patient at step 311 before a session is established by while the MA is online and connected to the doctor's electronic interface. Such preprocessing may include taking of vital signs like blood pressure, oxygen saturation, insulin levels, weight, patient complaints, or any other vitals that may be appropriate for the MA to take using portable medical equipment adapted to work with the MAs connected appliance.

In one embodiment, some medical appliances may be standalone devices that can be used to take information and the MA might input such data into the client application, which in turn may upload the information to the electronic interface for the attending physician before or during the appointment. Also, in step 311, the MA, having completed the pre-evaluation including vitals, etc. might manually set a flag for the appointment to ready. Such action communicates the state of the appointment directly to the physician's electronic interface relative to the appointment in queue. The physician then knows that the MA is ready to have the physician accept the appointment and establish a remote session. The patient information taken during step 311 may be uploaded to the electronic interface at step 312. Such data may include an indication icon or balloon associated visibly with the flag on the appointment icon resting in the visible queue.

In one embodiment, in addition to taking new vitals and initial information for upload to the physician for the current session, the MA (if medically authorized) may use the electronic interface sans physician to review, read, or interact with the patient's historical medical chart, which may include history of vital signs like temperature, blood Pressure, heart rate, pulse, the patient's chief complaint, history of the present illness, review of symptoms, such as those affecting the head, ears, eyes, nose, throat, cardiac signs, respiratory function, musculoskeletal information, condition of skin, condition of abdomen, genitourinary information, and pain. However, in a preferred embodiment, the physician has priority over access to and the ability to modify or change such patient data that will be part of the patient's record.

At step 313, the physician may select the icon associated with pre-evaluation or vitals to review the data before accepting the appointment. The physician may also access other information from other information sources connected to the network and review such data before accepting an appointment. After data review, the physician accepts the appointment at step 314, thus initiating a session over the network between the patient and MA and the physician. This may be accomplished by double clicking the appointment box or icon in queue. When the session legs are established and the connection or session is active, the system may set a new flag indicating that the appointment is currently in session at step 315. The new “in session” flag may replace the former “ready flag” of step 311. In this way all parties to the appointment queue, which could include more than one doctor operating from different electronic interfaces can see the current state for that appointment as being in session. In this way, session interruptions are less likely to occur.

During a session, it might be prudent for the physician to bring in or consult with third parties. The physician may determine, at step 316 whether to engage in third-party services on behalf of the patient. A third-party service might be adding a second physician associated with another facility for a supporting or consulting role. A third party might be a radiologist, a pharmacist, a lab technician, or another physician that will get a referral for one or more future appointments. In one embodiment, a session may include a team of specialists that may be conferring on behalf of the patient such as a tumor board deciding on a treatment strategy for a cancer patient currently in session with the oncologist. One or more third parties may be conference in using video conferencing software integrated with the electronic interface. Such conferencing may be performed in session with or without transparency too the patient.

If at step 316, the physician determines not to add any third-party services, the session continues and the system records and syncs (where applicable) all of the session data and available new records at step 318. Such documentation occurs automatically through the data session as new data and files become apparent. In one embodiment, recorded audio from the patient, MA, and doctor rendered as one or more text transcripts that are later retrievable. At step 316 if the physician determines to bring in third-party services, then at step 317 the physician may initiate such services. The process then resolves to step 318 and the session information and interaction data is recorded. It is important to mention that a session may be conducted between a patient and a doctor where the MA either drops out of the session to pre-evaluate the next patient or simply participates until the patient and doctor are comfortable. Moreover, a session may include family members or other support personnel at the patient location.

In other embodiments, the MA and physician may review patient's history, medical charts, and evaluation questions and answers together with or without the active participation of the patient including review of any current or historical laboratory results like blood. Urine, EKG (report and actual tracing), biologic cultures, X-rays, and on-site lab results like glucose, urinalysis, strep test, O2 saturation, and pregnancy test.

At step 319, the physician may determine to end the current session and move to accept a next session flagged ready for initiation. If at step 319 the physician determines to end the current session, a flag may be set at step 325 to indicate that the session is complete. The process may then resolve back to step 313 where the physician selects a next appointment icon to review a next patient's data, the process looping for each session. If the physician determines not to end a session, the process moves to step 320 and the current session is continued, and the remaining session data is recorded and synced at step 318. In any case, when a session ends, the remaining data not yet recorded is recorded including the time of session close. Other data may be included in closing data such as total time the session lasted.

In a typical session, the physician identifies himself to the patient and MA. The MA may conform the identification of the attending physician. The physician may also confirm the identification of the MA. The MD may also confirm the patient's identification. In one embodiment of the invention, a physician may order third party services such as new scans, blood work, cultures biopsies, prescribe medications, and make other orders that involve third-party or onsite (physician's facility) medical services. If during a session a patient experiences sudden health problems that cannot be resolved at the patient's location, the physician may order an ambulance and make a referral to a local hospital where adequate physicians and equipment are available such as through emergency services (ER).

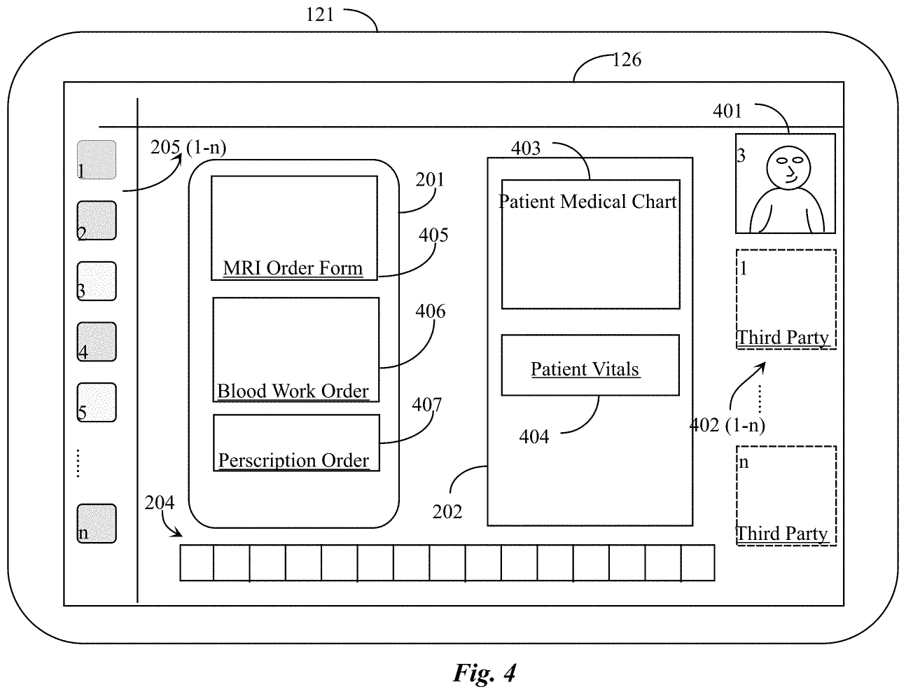

FIG. 4 is an exemplary screen shot of the electronic interface 126 of FIG. 1 including appointment and third-party session queues according to an embodiment of the present invention. Electronic interface 126 is shown in display on device screen 121. Interface 126 is, in a preferred embodiment, personalized for a physician that attends remote appointments over a network such as the Internet. Interface 126 includes data/graphics windows 201 and 202. Each window may display one or a number of items that may be read only items or items that may be edited by the physician. In this example, window 201 contains an interactive magnetic resonance imaging (MRI) order form 405.

MRI form 405 contains form fillable lines and is electronically signable. A physician may, during a session, call up form 405 fill it out and sign it during the session. The form may then be electronically forwarded to a third party by electronic message such as email or by electronic fax. The patent may also receive the form through the MA facilitating the session and the form can be printed at the location of the patient using patient equipment or a portable printer provided by the MA. Order forms for any other radiologic procedures may be initiated, filled out and delivered in the same manner as described above.

Window 201 contains a blood work order form 406. Blood work order 406 is an interactive electronic form that may also be filled out and signed by the physician. Form 406 may be delivered to the patient and printed out at the patient location. Window 201 contains a prescription order 407 for medication. Prescription order form 407, like other electronic forms, may be filled out and delivered by the physician during the interactive session. The physician may fill out form 407 and electronically mail or fax the order to the pharmacy on file for the patient or to any other pharmacy that might be determined based on the patient's location and level of transportation capability. In one embodiment, if the patient is located in a facility that has a pharmacy, the order may be electronically faxed to the pharmacy in the institution that is housing the patient.

Window 202 contains a patient's medical chart 403. Medical chart 403 may be any type of electronic text document with graphics as required to describe the patient demographics, medical conditions, possible allergies to medications, and current medications the patient is taking. A medical chart may be initiated by the physician during a session or before accepting the session if prior review of information is desired before talking with the patient. Window 202 also contains a chart 404 detailing the patient's current vital signs as taken by the MA before beginning the appointment session. The MA may forward patient vitals and other like information about the patient into the electronic interface where the data is associated with a session icon in queue before the physician accepts the appointment.

The physician may right click, for example, on an appointment icon and see via drop down menu, the documents that are available to the physician for review without requiring the physician to accept the appointment and initiate the session. This state is possible because the MA has access to the electronic interface for uploading the data regardless of whether the physician is currently using the interface to interact. This gives the physician time to review them before beginning the session. The electronic interface may reside on the physician's personal device or desktop. The electronic interface may instead reside on a sever where one or more physicians may or may not be working within the electronic interface. Window 201 and or 202 may also contain multimedia such as video clips taken of the patient, slide shows of pictures taken of the patient, or third-party multimedia information that can be displayed in either or both windows for educational purposes (for the patient and or MA to view).

In this example, appointment icons 205 (1-n) are arranged in a queue and displayed in the sidebar of electronic interface 126. In a preferred embodiment a non-physician assistant or MA facilitates the initiation of a session at the patient location by brining the required medical equipment (medical device peripherals) and network-capable appliance, and pre-staging the patient before the remote physician accepts the appointment. Pre-staging may include taking patient vital signs and filling out forms such as questionnaires about current conditions, pain levels, past surgeries, allergies to medications, and general information. All of the pre-staging data may be uploaded to a server and stored in a data repository that us accessible to the electronic interface. In one embodiment, the data at the network appliance of the MA at the patient's location may be “served” to the electronic interface on demand by the physician by right clicking on any of icons 205 (1-n). In another embodiment, the MA appliance and the physician's appliance sync data so that the patient has the correct information to review and the physician has the correct information to review.

Icons 205 (1-n) may be flagged or present in color according to a pre-determined color scheme that enables visual indication of the current state or stage of the appointment. In this example, there are two appointments in queue that are in session simultaneously. These are appointment 3 and appointment 5 in this example. Appointment 3 is colored yellow, in this example, indicating that the appointment is currently in session with a physician using electronic interface 126. A real-time video 401 of the MA for the patient is presented at the far-right corner of the interface. The same video may also show the patient instead of the MA or both the patient and the MA. The patient and MA may switch places in video 401 when required. More detailed video or pictures may be taken of the patient condition such as a wound, rash, bite, or any other physical abnormality that the physician might evaluate through high-resolution video.

Appointment number 5 is also yellow indicating that patient 5 is in session with a physician. In one embodiment, there may be two or more physicians working the queue containing icons 205 (1-n). Each physician would operate with a personalized version of the interface so the physicians would not see each other's patient data. However, the shared appointment queue is visible in the side bar of each personalized version of the physician's interface. In this case, data about patient number 5 is visible in the interface of the other physician currently running appointment 5. Appointment number 1 in the queue is grayed out indicating that the appointment has been completed. It may drop out of queue shortly after or immediately after it is terminated or closed.

Color-coding the appointment icons or setting visual flags aids physicians when working their own queue or a shared queue of appointments. For example, a physician would select a green appointment icon to initiate a fresh session. Icons 2, 4, and n are color-coded or flagged green indicating that the patients are ready to begin a remote session. Appointments in queue may be run in sequential order or worked, as they appear ready for session. For example, two patients may have 9AM appointments where one is successfully pre-processed for session before another so the green flag would indicate that state. Another color such as blue, for example, may be used to represent an appointment that is still being pre-processed by an MA. The color red may be used to indicate a medical urgency or priority. Appointments may be run by priority and by one or more physicians using personalized versions of the interface without departing from the spirit and scope of the present invention.

In this example, electronic interface 126 includes several third-party video windows 402 (1-n). Third-party windows 402 (1-n) enable physicians to teleconference with third parties while in session with a patient and MA. Link bar 204 may contain video conference call numbers that enable the physician to call third party practitioners including other physicians, radiologists, pharmacists, surgeons, other family members of the patient, and so on. In one embodiment, link bar 204 is somewhat personalized to the patient in session. For example, the third-party contacts that the physician might bring into a session through videoconference would be those that would be relevant to the patient and/or patient's condition and insurance setup. For example, if the physician attending the patient via remote session is the patient's oncologist, a third-party relevant to the patient may be the patient's primary care physician. The relevant third-party links may appear in tool bar or link bar 204 when the physician initiates the session.

In one embodiment, videoconferences may be initiated during a remote session with a patient and MA where the event is scheduled to occur during the session. In this embodiment, a team of physicians may join the session with the attending physician for the purpose of evaluating a complex case, giving second opinions, setting up other appointments, and so on. In one embodiment, the physician executes his or her electronic interface from his or her personal appliance such as an iPad™. The MA may execute his or her own application with a similar interface at the patient location. A server analogous to a game server or cloud server may then be utilized to sync the activities and interactions performed at each interface similar to a gaming environment. Data and files from the patient location may be uploaded to the server and then accessed by the physician and the MA may access files and data from the physician. In a preferred embodiment, the interaction between the physician and patient/MA is in real time during the remote session.

In one embodiment using the color-coding schema, a physician may set the color of an appointment icon or a flag associated with the icon to purple, for example, to indicate the session is active but the physician has left the session temporarily and will be back shortly. In one embodiment, pre-determined text indications are used to flag appointment icons instead of colors. One physician may also attend more than one remote session simultaneously by toggling back and forth between open sessions. In all embodiments, appropriate security protocols are observed such as authentication and login requirements, secure socket layer (SSL), virus protection, and data encryption where required.

Generally speaking, all of the functions a doctor may perform in a conventional setting, seeing patients in treatment rooms at a doctor's office, an urgent care facility or at an emergency room, the doctor may perform with the help of the MA and portable equipment in embodiments of the present invention. The doctor, in embodiments of this invention, however, is no longer “horizon limited”, and he or she may work far more efficiently and effectively. Moreover, the interaction occurring through a digital medium allows all activity and procedures to be recorded in real time with data backup, and for the doctor to have immediate access to internet-connected and local network connected information sources.

A doctor may see more patients and provide service to a greater number of people over time. By the virtue of Internet connectivity, doctors may operate in parts of the world where expenses are less than many other regions, and doctors may also specialize. The appointments web site doctors may be prioritized by skill and specialization as appointments are scheduled. A particular benefit of systems in embodiments of the present invention is in provision of full medical services to inmates of prisons and jails, and to residences of nursing homes and other residential facilities. It is well known that transporting such persons to an emergency room or an urgent care facility can be very expensive, and in some cases dangerous to the patient and/or the public.

The skilled person will understand and be aware that the elements of the invention and their interconnection and interaction in various embodiments may differ from the examples provided and described in a variety of ways, but still be within the spirit and scope of the invention. For example, there are many choices that may be made in vehicles that may be dispatched to provide Internet or more direct communication between remote sites where patents may interact with the system. There are variations in the sort of communication systems, which may, for example be Bluetooth™, WiFi, Satellite and other sorts. There are a wide variety of portable medical devices that a MA may use in the field and a wide variety of ways such devices may be used and may communicate information and data.

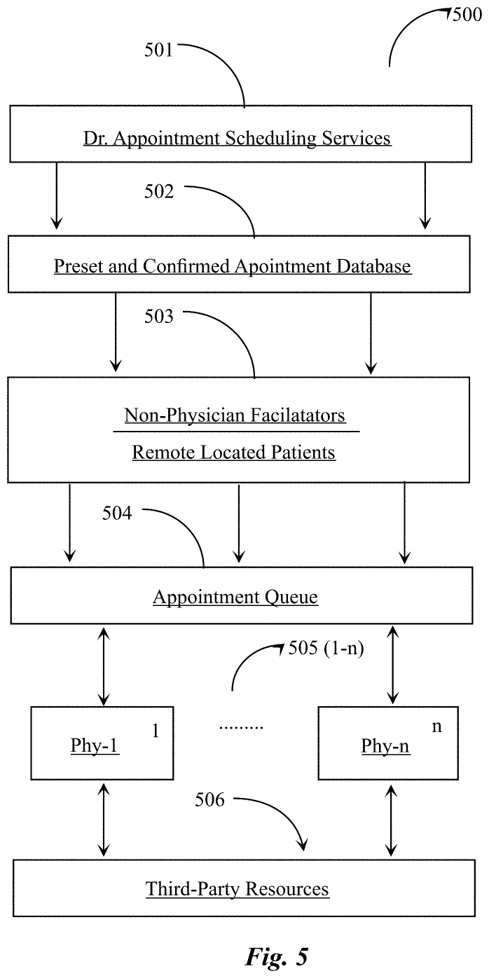

FIG. 5 is a process interaction chart 500 depicting patient services interaction according to a further embodiment of the present invention. In one embodiment a third-party service 501 may provide doctor appointment scheduling services for patients and facilities. Such services may help find insurance approved physicians for patients and further initiate scheduling of remote appointments. Block 502 underscores the activity of the service in presetting and then later confirming all appointments, such appointments stored on the network in a database or repository for later distribution and sync operations.

Non-physician assistants such as MAs or RNs may access preset and confirmed appointment lists for a work period. The service may utilize mapping services and GPS location information to help plan an efficient route for non-physician assistants to “run” the appointments. Block 503 represents the non-physician facilitators at the remote patient locations. An MA may at the start of a workday download his or her appointments for that day. Travel time and other break periods are worked into the schedule so that the MA is not over-utilized or under-utilized with respect to the number of locations (patients) visited. One with skill in the art will appreciate that if the MA is dispatched to a facility where all of the day's patients are facilitated, the MA may take more patients because there is no travel time from location to location.

Block 504 represents an appointment queue that appears in the physician's electronic interface. In one embodiment, one or more MAs may be assigned to a physician and those preset and confirmed appointments show up in queue on the scheduled day of service. Visual indication techniques such as flagging enables the physician to see which appointments are ready for a session and which are not yet ready to begin. An MA and patient may experience a waiting period between an indication of ready (flag set by MA) and when the physician accepts the session similar to a waiting room at a clinic. In this regard, music may be played over the MA interface including advertising. The queue may include an estimated waiting time (EWT) for a physician to respond. The EWT may be communicated from the system to the MA interface before the appointment is accepted.

Physicians 505 (1-n) represent physicians who may work a queue of appointments in an embodiment of a shared appointment queue. There is some advantage to having more than one physician share a queue in that more appointments can be conducted and completed in a smaller amount of time. Scenarios may include an office where more than one primary care physician is approved for seeing any one patient similar to a walk-in clinic where the patient does not know which of the onsite doctors will see them. On the other hand, there may be just one physician working a queue set up for that physician. Physicians are not required to be onsite at a physical medical facility in order to take appointments. In one embodiment, a physician using an iPad™ or another hand-held or portable network-capable appliance may connect online, access the electronic interface, and run appointments from home or from another location. Patient records, and other source data required in a session may be accessed from a computing cloud service or from a connected server.

Block 506 represents a third-party resource bar analogous to link bar 204 of FIG. 2. In one embodiment, there may be a mix of links or addresses or numbers, some of which connect to patient neutral third parties and some of which may connect to patient centric third parties. Patient neutral parties may include new physicians or other medical practitioners that the patient may consult with and may or may not set an appointment with. Patient centric third parties might include the patient's boss, family member or members, physicians known to and previously used by the patient, etc. The resource bar may also include scientific and medical news, latest study data, or other resources that may be used by and shared among medical professionals. There are many possibilities.

It will be apparent to one with skill in the art that the remote medical patient servicing system of the invention may be provided using some or all of the mentioned features and components without departing from the spirit and scope of the present invention. It will also be apparent to the skilled artisan that the embodiments described above are specific examples of a single broader invention that may have greater scope than any of the singular descriptions taught. There may be many alterations made in the descriptions without departing from the spirit and scope of the present invention.

What is claimed is:

1. A system comprising:
a computerized appliance having a processor executing software, the computerized appliance connected to the Internet network;
a data repository coupled to the computerized appliance;
a display monitor coupled to the computerized appliance;
a portable communication device having audio and imaging apparatus, connected to the Internet network, and located at a at a patient's geographic location remote from the computerized appliance;
an interactive interface displayed on the display monitor by execution of the software by the processor; and
a plurality of icons displayed in a chronological queue in the interactive interface, each icon representing an appointment with a patient, each icon comprising first indicia providing identification of the patient, second indicia providing date and time of day for the associated appointment, and third indicia providing real-time status of the appointment, as ready and waiting, in progress or finished;
characterized in that a physician user at the computerized appliance, by virtue of execution of the software by the processor, is enabled to interact with an individual one of the icons indicated as a waiting appointment, such that selecting the icon initiates a video conference between the computerized appliance and the portable communication device and updates the status of the appointment to in progress.

2. The system of claim 1 wherein the physician user, engaged in a video conference with the portable communication device at the patient's geographic location, is enabled to access medical records associated with the patient, and to display such records on the display monitor.

3. The system of claim 2 wherein the patient's medical records are downloaded from an Internet site.

4. The system of claim 1 wherein the physician user is enabled, through the software execution, to rearrange the appointment icons as to date and time of day.

5. The system of claim 1 wherein the indicia providing status is a tag having a color indicating the status.

6. The system of claim 1 further comprising a local area network (LAN), the computerized appliance being a first appliance connected to the LAN, further comprising second computerized appliances with display monitors also connected to the LAN, enabling a plurality of physician users to conduct video conferences with second portable communication devices at patient's geographic locations.

7. The system of claim 1 wherein a medical assistant is sent to the patient's geographic location near the time of an appointment, and the medical assistant operates the portable communication device in a video conference with a physician user.

8. The system of claim 1 further comprising a medical measuring device coupled to the portable communication device, the medical measuring device measuring a physical characteristic of the patient and communicating the measurement to the computerized appliance over the Internet network.

9. The system of claim 1, wherein the computerized appliance is one of a smart phone, a laptop computer, a notebook computer, or a pad device.

10. The system of claim 1 further comprising functionality enabled by execution of the software enabling persons operating Internet connected computer devices to access the computerized appliance over the Internet network, and to request or cancel an appointment.

11. A method comprising:
displaying a plurality of icons in a chronological queue in an interactive interface on a display monitor coupled to a computerized appliance connected to the Internet network and having a coupled data repository, each icon representing an appointment with a patient, with each icon comprising first indicia providing identification of the patient, second indicia providing date and time of day for the associated appointment, and third indicia providing real-time status of the appointment, as ready and waiting, in progress or finished; and
interacting with an individual one of the icons by a physician user, initiating a video conference between the computerized appliance and a portable communication device having audio and imaging apparatus, located at a geographic location of the patient remote from the computerized appliance, initiating a video conference between the computerized appliance and the portable communication device and updating the status of the appointment to in progress.

12. The method of claim 11 wherein the physician user, engaged in a video conference with the portable communication device at the patient's geographic location, accesses medical records associated with the patient, and displays such records on the display monitor.

13. The method of claim 12 comprising downloading the patient's medical records from an Internet site.

14. The system of claim 11 comprising the physician user rearranging the appointment icons as to date and time of day.

15. The method of claim 11 comprising providing status of an appointment by a tag having a color indicating the status.

16. The method of claim 11 further comprising enabling a plurality of physician users to interact with appointment icons and initiate video conferences by connecting a plurality of computerized appliances having connected displays and data repositories to a local area network (LAN).

17. The method of claim 11 comprising sending a medical assistant to the patient's geographic location near the time of an appointment, the medical assistant operating the portable communication device in a video conference with a physician user.

18. The method of claim 11 further comprising coupling a medical measuring device to the portable communication device, measuring a physical characteristic of the patient by the medical measuring device and communicating the measurement to the computerized appliance over the Internet network.

19. The method of claim 11, wherein the computerized appliance is one of a smart phone, a laptop computer, a notebook computer, or a pad device.

20. The method of claim 11 further comprising accessing the computerized appliance over the Internet network by a person operating an Internet connected computer device, requesting or cancelling an appointment.
